(19) United States
(12) Patent Application Publication
(10) Pub. No.: US 2006/0270044 A1
(43) Pub. Date: Nov. 30, 2006

(54) Compositions and methods for altering cellular functions

(75) Inventors: Hideki Suzuki, Madison, WI (US); Donna M. Bates, Madison, WI (US)

Correspondence Address: Medlen & Carroll, LLP, Suite 350, 101 Howard Street, San Francisco, CA, 94105, US

(73) Assignee: ConjuGon, Inc., Madison, WI (US)

(21) Appl. No.: 11/137,948

(22) Filed: May 26, 2005

(51) Int. Cl. C12N 15/74; C12N 1/21

(52) U.S. Cl. 435/471; 435/473; 435/252.3

(57) ABSTRACT

The present invention relates to the field of bacteriology. In particular, the invention relates to novel compositions and methods for altering (e.g., inhibiting) the growth and virulence of populations of pathogenic microorganisms.

### FIELD OF THE INVENTION

The present invention relates to the field of bacteriology. In particular, the invention relates to novel compositions and methods for altering (e.g., inhibiting) the growth and virulence of populations of pathogenic microorganisms.

### BACKGROUND OF THE INVENTION

As the use of conventional pharmaceutical antibiotics (herein referred to as antibiotics) increases for medical, veterinary and agricultural purposes, there has been a concurrent emergence of antibiotic-resistant strains of pathogenic bacteria. This has become of major concern inasmuch as drug resistance of bacterial pathogens is presently the major cause of failure in the treatment of infectious diseases (e.g., Staphylococcus pneumoniae, causing meningitis; Pseudomonas aeruginosa, causing pneumonia; and Mycobacterium tuberculosis, causing tuberculosis).

The emergence of single- or multi-drug resistant bacteria results from a gene mobilization that responds quickly to the strong selective pressure that is a consequence of antibiotic use. Over the last several decades, the increasingly frequent usage of antibiotics has acted in concert with spontaneous mutations arising in the bacterial gene pool to produce antibiotic resistance in certain strains. This is particularly true for opportunistic pathogens that are most often acquired nosocomially (e.g., in hospitals or other settings where antibiotics are routinely used). This gene pool is continually utilized by previously sensitive strains capable of accessing it by various means including the transfer of extrachromosomal elements (plasmids) by conjugation. As a result, single- and multi-drug resistance genes are commonly found in a large variety of bacterial plasmids.

Presently there is no known method by which to avoid the selection of antibiotic resistant bacterial mutants that arise as a result of the many standard applications of antibiotics in the modern world. Furthermore, infections caused by such pan-resistant strains of microorganisms can be extremely difficult to treat with the current arsenal of antimicrobial agents. Accordingly, a great need exists to develop alternative strategies of antibacterial treatment.

Interest in the use of bacteriophages to treat infectious diseases developed early in the twentieth century, and has undergone resurgence in recent years. For example, bacteriophages have been shown to be effective in the treatment of certain pathogenic E. coli species in laboratory and farm animals, and have been proposed as a viable alternative to the use of antibiotics (Smith & Huggins, J. Gen. Microbiol. 128: 307-318, 1981; Smith & Huggins, J. Gen. Microbiol. 129: 26592675, 1983; Smith et al., J. Gen. Microbiol. 133: 1111-1126, 1986; ICuvda et al., Appl. Env. Microbiol. 65: 3767-3773, 1999). However, the use of bacteriophages as antimicrobial agents has certain limitations. First, the relationship between a phage and its host bacterial cell is typically very specific, such that a broad host-range phage agent generally is unavailable. Second, the specificity of interaction usually arises at the point of the recognition and binding of phage to the host cell. This often occurs through the expression of surface receptors on the host cell to which a phage specifically binds. Inasmuch as such receptors are usually encoded by a single gene, mutations in the host bacterial cell to alter the surface receptor, thereby escaping detection by the phage, can occur with a frequency equivalent to or higher than, the mutation rate to acquire antibiotic resistance. As a result, if phage were utilized as commonly as antibiotics, resistance of pathogenic bacteria to phages could become as common a problem as antibiotic resistance.

Another approach to controlling pathogenic bacteria has been proposed, which relies on using molecular biological techniques to prevent the expression of antibiotic resistance genes in pathogenic bacteria (see, e.g., U.S. Pat. No. 5,976,864).

In this method, a nucleic acid construct encoding an “external guide sequence” specific for the targeted antibiotic resistance gene is introduced into the pathogenic bacterial cells. The sequence is expressed, hybridizes with messenger RNA (mRNA) encoding the antibiotic resistance gene product, and renders such mRNA sensitive to cleavage by the enzyme RNAse P. Such a system also has limited utility, since it is targeted to specific antibiotic resistance genes. While the system may be effective in overcoming resistance based on expression of those specific genes, continued use of the antibiotics places selective pressure on the bacteria to mutate other genes and develop resistance to the antibiotic by another mechanism.

Microorganisms continue to evolve to evade current treatments. It is clear that current alternatives to antibiotic use are limited and suffer many of the same drawbacks as antibiotic use itself. Thus a great need exists to develop alternative strategies of antibacterial treatment.

### SUMMARY OF THE INVENTION

The present invention relates to the field of bacteriology. In particular, the invention relates to novel compositions and methods for altering (e.g., inhibiting) the growth and virulence of populations of pathogenic microorganisms.

Accordingly, the present invention provides a method of treating a recipient cell, comprising conjugatively transferring into the recipient cell a recombinant transmissible plasmid configured to alter the virulence of the recipient cell. In preferred embodiments, the recipient cell is a pathogen cell, and the altering the virulence of the recipient cell comprises attenuating the virulence of the pathogen cell. In some embodiments, the conjugatively transferring comprises exposing the recipient cell to a donor cell, wherein the donor cell comprises the recombinant transmissible plasmid, and wherein the donor cell is configured to conjugatively transfer the recombinant transmissible plasmid to a recipient cell. In some embodiments, the donor cell comprises one or more transfer genes. In some preferred embodiments, the one or more transfer genes are contained on the recombinant transmissible plasmid.

In some embodiments, the recipient cell is a Gram-negative bacterium, while in other embodiments the recipient cell is a Gram-positive bacterium. In preferred embodiments, the recipient cell is of a genus selected from, but not limited to, Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium, Treponema, Borrelia, Leptospira, and Chlamydiae. In some embodiments, the microorganism is selected from the group consisting of Vibrio harveyi, Vibrio cholerae, Vibrio parahaemolyticus, Vibrio alginolyticus, Pseudomonas phosphoreum, Yersinia enterocolitica, Escherichia coli, Salmonella typhimurium, Haemophilus influenzae, Helicobacter pylori, Bacillus subtilis, Borrelia Burgdorferi, Neisseria meningitidis, Neisseria gonorrhoeae, Yersinia pestis, Campylobacter jejuni, Deinococcus radiodurans, Mycobacterium tuberculosis, Enterococcus faecalis, Streptococcus pneumoniae, Streptococcus pyogenes and Staphylococcus aureus.

In some embodiments, the donor cell is a bacterium. In other embodiments, the donor cell is a non-dividing cell.

In some embodiments, the recombinant transmissible plasmid comprises nucleic acid encoding at least one dominant negative form of a protein involved in a secretion system. In preferred embodiments, the secretion system is selected from a group consisting of type II, type III, and Type IV secretion systems. In some embodiments, the recombinant transmissible plasmid is selected from the group consisting of pCON22-98, pCON22-35 and pCON22-62.

In some embodiments, the nucleic acid encodes a pscN protein comprising a dominant negative mutation. In some embodiments, the dominant negative mutation in the pscN protein is a K176G mutation. In some embodiments, the nucleic acid encodes an xcpR protein comprising a dominant negative mutation. In some embodiments, the dominant negative mutation in the xcpR protein is a G268A mutation. In some embodiments, the nucleic acid encodes dominant negative pscN and dominant negative xcpR proteins. In some preferred embodiments, the nucleic acid encodes a protein comprising at least one mutation in a Walker box A motif.

In some embodiments, the present invention also provides a composition comprising a donor cell comprising a plasmid, wherein the plasmid comprises nucleic acid sequence encoding a dominant negative form of a protein involved in type II, type III, or Type IV secretion systems. In some embodiments, the plasmid is self-transmissible. In some embodiments, the nucleic acid sequence encodes dominant negative pscN. In some embodiments, the nucleic acid sequence encodes a K176G dominant negative form of pscN. In some embodiments, the nucleic acid sequence encodes dominant negative xcpR. In some embodiments, the nucleic acid sequence encodes a G268A dominant negative form of xcpR. In some embodiments, the nucleic acid sequence encodes both dominant negative pscN and dominant negative xcpR. In some embodiments, the nucleic acid sequence encodes Walker box A mutations. In some embodiments, the transmissible plasmid is pCON22-98, pCON22-35 or pCON22-62. In some embodiments, the transmissible plasmid pCON4-78 is used to generate the plasmid comprising nucleic acid sequence encoding a dominant negative form of a protein involved in type II or type II secretion systems.

In some embodiments, the recombinant transmissible plasmid comprises nucleic acid encoding a protein that inhibits a regulatory protein involved with a secretion system, e.g., such that the function of the secretion system is disrupted. In preferred embodiments, the secretion system is selected from a group consisting of type II, type III, and Type IV secretion systems.

The method of Claim 13, wherein said secretion system is selected from a group consisting of type II, type III, and Type IV secretion systems.

The present invention also provides systems comprising a material in contact with a donor cell of the present invention, e.g., comprising a recombinant transmissible plasmid, wherein the recombinant transmissible plasmid comprises nucleic acid sequence encoding at least one dominant negative form of a protein involved in a secretion system, and wherein the donor cell is configured to conjugatively transfer said recombinant transmissible plasmid to a recipient cell. In some embodiments, the secretion system is selected from the group consisting of type II, type III and Type IV secretion systems. In some embodiments, the material of the system comprises a medical device. In other embodiments, the material comprises a pharmaceutically acceptable compound.

It is contemplated that the methods and compositions of the present invention may be used to treat surfaces. Surfaces that can be treated by the methods and compositions of the present invention include but are not limited to surfaces of a medical device, a wound care device, a body cavity device, a human body, an animal body, a personal protection device, a birth control device, and a drug delivery device. Surfaces include but are not limited to tissues such as skin and other soft tissue, bone, silicon, plastic, glass, polymer, ceramic, photoresist, skin, tissue, nitrocellulose, hydrogel, paper, polypropylene, cloth, cotton, wool, wood, brick, leather, vinyl, polystyrene, nylon, polyacrylamide, optical fiber, natural fibers, nylon, metal, rubber and composites thereof. In some embodiments, the treating reduces the virulence of recipient cells on the surface, while in other embodiments the treatment attenuates recipient cells that come into contact with the surface after the treatment. see, e.g., co-pending patent application filed May 26, 2005, under Express Mail Label No. EV618124316US, which is incorporated herein by reference in its entirety for all purposes.

### DESCRIPTION OF THE DRAWINGS

FIG. 1 displays the virulence of P. aeruginosa PA14 in a murine pneumonia model. The lowest two dilutions are overlapping in the graph.

FIG. 2 shows bacterial virulence is not affected by carrying wild-type RK2 plasmid.

FIG. 3 shows bacterial virulence is significantly reduced by dominant-negative mutant genes.

FIG. 4 shows T2 and T3 mutants, individually, have a significant effect on bacterial virulence.

FIG. 5 shows that a pathogen carrying the mutant genes displays attenuated virulence in a different animal breed.

FIG. 6 depicts the plasmid pCON4-78.

FIG. 7 depicts the plasmid pCON22-98.

FIG. 8 depicts the plasmid pCON22-35.

FIG. 9 depicts the plasmid pCON22-62.

### DEFINITIONS

To facilitate an understanding of the present invention, a number of terms and phrases are defined below:

As used herein, the term “subject” refers to individuals (e.g., human, animal, or other organism) to be treated by the methods or compositions of the present invention. Subjects include, but are not limited to, mammals (e.g., murines, simians, equines, bovines, porcines, canines, felines, and the like), and most preferably includes humans. In the context of the invention, the term “subject” generally refers to an individual who will receive or who has received treatment (e.g., administration of donor cell, and optionally one or more other agents) for a condition characterized by the presence of pathogenic bacteria, or in anticipation of possible exposure to pathogenic bacteria.

The term “diagnosed,” as used herein, refers to the to recognition of a disease (e.g., caused by the presence of pathogenic bacteria) by its signs and symptoms (e.g., resistance to conventional therapies), or genetic analysis, pathological analysis, histological analysis, and the like.

As used herein the term, “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reaction that occur within a natural environment.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “conjugation” refers to the process of DNA transfer from one cell to another. Although conjugation is observed primarily between bacterial cells, this process takes place from bacterial cells to higher and lower eukaryote cells (Waters, Nat Genet. 29:375-376 [2001]; Nishikawa et al., Jpn J Genet. 65:323-334 [1990]). Conjugation is mediated by complex cellular machinery, and essential protein components are often encoded as a series of genes in a plasmid (e.g., the tra genes for plasmid RK2). Some of these gene products are assembled to facilitate a direct cell-cell interaction (e.g., mating pair formation), and some of them serve to transfer DNA and associated protein molecules, and to start replication of the DNA molecule (e.g., DNA transfer/replication). oriT is a DNA sequence from which the transfer of a DNA molecule initiates in the process of conjugation.

As used herein, the terms “conjugation donor” and “donor cell” are used interchangeably to refer to a cell, e.g., a bacterial cell, carrying a plasmid, wherein said plasmid can be transferred to another cell through conjugation. Examples of donor cells include, but are not limited to E. coli strains that contain a self-transmissible plasmid or a non-self-transmissible plasmid. A cell receiving a plasmid or other cellular material from a donor cell via conjugative transfer is referred to as a “recipient cell”. As used herein, the term “transmissible plasmid” refers to a plasmid that can be transferred from a donor cell to a recipient cell via conjugation.

As used herein, the term “self-transmissible plasmid” refers to a plasmid encoding all the genes needed to mediate conjugation. A recipient of a self-transmissible plasmid becomes a proficient donor to further transfer the self-transmissible plasmid to another recipient cell.

As used herein, the term “non-self-transmissible plasmid” or “mobilizable plasmid” refers to a plasmid lacking some of the genes needed to mediate conjugation. A cell carrying a non-self-transmissible plasmid does not transfer DNA through conjugation unless the missing gene(s) are supplied in trans within the same cell. Therefore, a recipient cell that lacks the missing gene(s), does not become a proficient conjugation donor when it receives the non-self-transmissible plasmid.

As used herein, the term “origin of transfer” or “oriT” refers to the cis-acting site required for DNA transfer, and integration of an oriT sequence into a non-transmissible plasmid converts it into a mobilizable plasmid (Lanka and Wilkins, Annu Rev Biochem, 64:141-169 [1995]).

In some embodiments, a donor cell is a bacterial cell (e.g., a Gram-positive or Gram-negative bacterium). Examples of donor cells include, but are not limited to, bacterial cells of a genus of bacteria, selected from the group consisting of Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium, Treponema, Borrelia, Leptospira, and Chlamydiae.

In some embodiments, a donor cell is a non-viable cell, including but not limited to a bacterial minicell, a maxicell, or a non-dividing cell.

As used herein, the term “maxicell” refers to the cells that have been treated to maximize chromosomal degradation, e.g., by UV irradiation and extended incubation. Maxicells contain mostly plasmid DNA, and synthesis of proteins within maxicells occurs essentially exclusively from the plasmid DNA in the cells.

As used herein, the term “non-dividing cell” or “ND cell” refers to cells that are treated in a manner selected to preferentially damage the chromosomal DNA of the cell (e.g., by UV or other irradiation), wherein said cells are further treated, e.g., by rapid chilling after DNA damaging treatment, to minimize chromosomal degradation. “ND cells” can also be obtained in a process such as temporal expression of bactericidal protein (e.g., ColE3) within a donor bacterium. Thus, in some embodiments, induction of proteins (e.g., ColE3) destroys the protein synthesis in the cell, leading to cell death while leaving the conjugation apparatus and chromosomal DNA synthesized prior to ColE3 synthesis intact. ND cells contain both chromosomal and plasmid DNA but the function of the cell is sufficiently altered, e.g., by UV irradiation, that said ND cells have little or no capability to divide.

The terms “target cells,” “targets,” “recipient cells,” and “recipients” are used interchangeably herein. In preferred embodiments, the target cells for the compositions and methods of the present invention include, but are not limited to, pathogenic microorganisms (e.g., pathogenic bacteria). Pathogenic bacteria include, but are not limited to, Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium, Treponema, Borrelia, Leptospira, and Chlamydiae. In some embodiments, recipient cells are continuously cultured cells. In some embodiments the recipient cells are uncultured cells existing in their natural environment (e.g., at the site of a wound or infection) or obtained from patient tissues (e.g., via a biopsy). In one specific embodiment, the recipient cells exhibit pathological growth or proliferation.

As used herein, the terms “attenuate” and “attenuation” as used herein in reference to a feature e.g., of a target cell, refers to a reducing or weakening of that feature, or a reducing of the effect(s) of that feature. For example, when used in reference to a pathogen or the pathogenicity of a target cell, attenuation generally refers to a reduction in the virulence of the pathogen. Attenuation of a pathogen is not limited to any particular mechanism of reduced virulence. In some embodiments, reduced virulence maybe achieved, e.g., by disruption of a secretory pathway. In other embodiments, reduced virulence may be achieved by altering cellular metabolism to increase reactivity to or susceptibility to a drug, e.g., a drug that attenuates virulence of the pathogen, or that kills the pathogen. In some embodiments, attenuation refers to a feature, e.g., virulence of a population of cells. For example, in some embodiments of the present invention, a population of pathogen cells is treated, e.g., by the methods and compositions of the invention, such that the population of cells is decreased in virulence.

As used herein, the term “virulence” refers to the degree of pathogenicity of a microorganism, e.g., as indicated by the severity of the disease produced or its ability to invade the tissues of a subject. It is generally measured experimentally by the median lethal dose (LD₅₀) or median infective dose (ID₅₀). The term may also be used to refer to the competence of any infectious agent to produce pathologic effects.

As used herein, the term “effective amount” refers to the amount of a composition (e.g., donor bacteria cells) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., compositions of the present invention) to a physiological system (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

As used herein, the term “co-administration” refers to the administration of at least two agent(s) (e.g., two separate donor bacteria, each comprising a different plasmid) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “toxic” refers to any detrimental or harmful effects on a subject, a cell, or a tissue as compared to the same cell or tissue prior to the administration of the toxicant.

As used herein, the term “pharmaceutical composition” refers to the combination of an active agent (e.g., donor bacteria cells) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “topically” refers to application of the compositions of the present invention to the surface of the skin and mucosal cells and tissues (e.g., alveolar, buccal, lingual, masticatory, or nasal mucosa, and other tissues and cells that line hollow organs or body cavities).

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “medical device” includes any material or device that is used on, in, or through a subject's or a patient's body in the course of medical treatment (e.g., for a disease or injury). Medical devices include, but are not limited to, such items as medical implants, wound care devices, drug delivery devices, and body cavity and personal protection devices. The medical implants include, but are not limited to, urinary catheters, intravascular catheters, dialysis shunts, wound drain tubes, skin sutures, vascular grafts, implantable meshes, intraocular devices, heart valves, and the like. Wound care devices include, but are not limited to, general wound dressings, biologic graft materials, tape closures and dressings, and surgical incise drapes. Drug delivery devices include, but are not limited to, needles, drug delivery skin patches, drug delivery mucosal patches and medical sponges. Body cavity and personal protection devices, include, but are not limited to, tampons, sponges, surgical and examination gloves, and toothbrushes. Birth control devices include, but are not limited to, intrauterine devices (IUDs), diaphragms, and condoms.

As used herein, the term “therapeutic agent,” refers to compositions that decrease the infectivity, morbidity, or onset of mortality in a subject contacted by a pathogenic microorganism or that prevent infectivity, morbidity, or onset of mortality in a host contacted by a pathogenic microorganism. As used herein, therapeutic agents encompass agents used prophylactically, e.g., in the absence of a pathogen, in view of possible future exposure to a pathogen. Such agents may additionally comprise pharmaceutically acceptable compounds (e.g., adjutants, excipients, stabilizers, diluents, and the like). In some embodiments, the therapeutic agents of the present invention are administered in the form of topical compositions, injectable compositions, ingestible compositions, and the like. When the route is topical, the form may be, for example, a solution, cream, ointment, salve or spray.

As used herein, the term “pathogen” refers a biological agent that causes a disease state (e.g., infection, cancer, etc.) in a host. “Pathogens” include, but are not limited to, viruses, bacteria, archaea, fungi, protozoans, mycoplasma, prions, and parasitic organisms.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are Gram-negative or Gram-positive. “Gram-negative” and “Gram-positive” refer to staining patterns with the Gram-staining process, which is well known in the art. (See e.g., Finegold and Martin, Diagnostic Microbiology, 6th Ed., CV Mosby St. Louis, pp. 13-15 (1982)). “Gram-positive bacteria” are bacteria that retain the primary dye used in the Gram stain, causing the stained cells to generally appear dark blue to purple under the microscope. “Gram-negative bacteria” do not retain the primary dye used in the Gram stain, but are stained by the counterstain. Thus, Gram-negative bacteria generally appear red.

As used herein, the term “microorganism” refers to any species or type of microorganism, including but not limited to, bacteria, archaea, fungi, protozoans, mycoplasma, and parasitic organisms. The present invention contemplates that a number of microorganisms encompassed therein will also be pathogenic to a subject.

As used herein, the term “fungi” is used in reference to eukaryotic organisms such as the molds and yeasts, including dimorphic fungi.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). A polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences, or 5′ flanking sequences. Sequences located 3′ or downstream of the coding region and present on the mRNA are referred to as 3′ non-translated sequences or 3′ flanking sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into pre-mRNA; introns may contain regulatory elements such as enhancers. Introns are generally removed or “spliced out” from the primary (pre-mRNA) transcript; introns therefore are generally absent in the messenger RNA (mRNA) transcript. The mRNA functions during translation to specify the sequence or order of amino acids in a nascent polypeptide.

As used herein, the term “heterologous gene” and “heterologous nucleic acid” refers to a gene or nucleic acid that is not in its natural environment. For example, a heterologous gene or nucleic acid includes a gene or nucleic acid from one species introduced into another species. A heterologous gene or nucleic acid also includes a gene or nucleic acid native to an organism that has been altered in some way (e.g., mutated, added in multiple copies, linked to non-native regulatory sequences, etc). Heterologous genes or nucleic acids are distinguished from endogenous genes or nucleic acids in that the heterologous gene or nucleic acid sequences are typically joined to DNA sequences that are not found naturally associated with the gene or nucleic acid sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “wild-type” refers to a gene or gene product in the form that would be isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” “nucleic acid encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides such as an oligonucleotide or a target nucleic acid) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

The term “homology” refers to a degree of similarity between molecules such as nucleic acid molecules. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any nucleic acid that can hybridize to either or both strands of the double-stranded nucleic acid sequence under conditions of low stringency as described above. When used in reference to a single-stranded nucleic acid sequence, the term “substantially homologous” refers to any nucleic acid that can hybridize (i.e., it is the complement of) to the complement of the single-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.” As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample. In yet another example, nucleic acids in a sample are purified by removing or reducing one or more components from a sample. Components to be reduced or removed in purification comprise other nucleic acids, damaged nucleic acids, proteins, salts, etc.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “transdominant negative mutant gene” refers to a gene encoding a protein product that prevents other copies of the same gene or gene product, which have not been mutated (i.e., which have the wild-type sequence) from functioning properly (e.g., by inhibiting wild type protein function). The product of a transdominant negative mutant gene is referred to herein as “dominant negative” or “DN” (e.g., a dominant negative protein, or a DN protein).

As used herein, the term “kit” refers to any delivery system for delivering materials. In the context of reaction materials such as donor cells, such delivery systems include systems that allow for the storage, transport, or delivery of reaction reagents (e.g., cells, buffers, selection reagents, etc., in the appropriate containers) and/or supporting materials (e.g., media, written instructions for performing using the materials, etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain cells for a particular use, while a second container contains selective media. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction materials needed for a particular use in a single container (e.g., in a single box housing each of the desired components). The term “kit” includes both fragmented and combined kits.

As used herein, the term “cellular metabolic function” refers to any or all processes conducted by a cell (e.g., enzymatic or chemical processes associated with cell function), other than genomic replication.

### DETAILED DESCRIPTION OF THE INVENTION

Microorganisms (e.g., Gram-negative bacteria) cause a wide variety of diseases in humans. While some species are solely pathogenic, others (e.g., Pseudomonas aeruginosa), are opportunistic pathogens, causing disease only under certain circumstances. The ability of an opportunistic pathogen to cause disease is most often due to a compromise in host immune function and/or acquisition of new virulence factors on the part of the bacterium. Many of these opportunistic pathogens are acquired in hospitals or other settings where antibiotics are routinely used. Thus, these opportunistic pathogens are often resistant to many different types of antibiotics (e.g., pan-resistant).

Thus, in some embodiments, the present invention provides donor cells comprising a transmissible plasmid, wherein the plasmid may be transferred (e.g., through conjugation) from the donor cell to a recipient cell (e.g., a pathogenic microorganism), resulting in the plasmid expressing its genetic material in the recipient cell, so as to alter a cellular function, e.g., a virulence factor, of the recipient cell. In preferred embodiments, the transmissible plasmid is a recombinant transmissible plasmid. Conjugation for transferring genetic material from a donor cell into a target recipient cell for a variety of purposes has been described. See, e.g., PCT Publication WO 02/18605, U.S. Patent Application Ser. No. 20040137002, and U.S. patent application Ser. No. 10/884,257, each incorporated herein by reference in its entirety for all purposes. The present invention makes use of conjugative transfer to alter the cellular functions of a recipient cell, e.g., to attenuate virulence.

Gram-negative pathogens produce a variety of virulence factors, many of which are proteins that are secreted extracellularly. Although there are multiple pathways of protein secretion in Gram-negative bacteria (see, e.g., Sandkvist, Molecular Microbiology, 40(2): 271-283 (2001); Sandkvist, Infection and Immunity, 69(6): 3523-3535 (2001); Buttner and Bonas, Trends in Microbiology, 10(4): 186-192 (2002)) the majority of the virulence factors are secreted through either the type II or type III pathway.

Translocation of proteins through the type II system requires that the protein first enter the periplasm through the general secretory (sec) pathway (see, e.g., FEMS Microbiol Lett., 238(2):273-279 (2004)). Upon maturation of the protein by removal of an N-terminal signal sequence, it is translocated across the outer membrane by type II-specific components. Because of its sec-dependent nature, the type II system is often referred to as the main terminal branch of the general secretory pathway and it is found in all Gram-negative bacteria. Examples of protein virulence factors that are secreted by this system include proteases, cellulases, pectinases, phospholipases, lipases, and toxins (see, e.g., Sandkvist, Molecular Microbiology, 40(2): 271-283 (2001)).

The type II system is highly sophisticated, consisting of a multitude of proteins, encoded by at least 12 different genes (see, e.g., Sandkvist, Molecular Microbiology, 40(2): 271-283 (2001)). The components of this system assemble within the periplasm and outer membrane and evidence suggests that they interact with one another through multiple protein-protein interactions (see, e.g., Kagami et al., Molecular Microbiology 27(1): 221-233 (1998); Ball et al., Journal of Bacteriology 181(2): 382-388 (1999); Sandkvist et al., Journal of Bacteriology 182(3): 742-748 (2000); Possot et al., Journal of Bacteriology 182(8): 2142-2152 (2000)). The components of the type II system are similar to those of the bacterial type IV pilus biogenesis system and the two systems may function similarly in the export of their specific substrates (i.e. secreted proteins for type II system, pilins for type IV pilus biogenesis) (see, e.g., Sandkvist, Molecular Microbiology, 40(2): 271-283 (2001); Peabody et al., Microbiology 149: 3051-3072 (2003)).

In contrast to the ubiquitous presence of the type II system in all Gram-negative bacteria, the type III secretion system (TTSS) appears to be specific to pathogens (see, e.g., Hueck, Microbiology and Molecular Biology Reviews 62(2): 379-433 (1998); Cornelis, Anuual Review of Microbiology 54: 735-774 (2000); Plano and Ferracci, Molecular Microbiology 40(2): 284-293 (2001); Buttner, Trends in Microbiology 10(4): 186-192 (2002); Gauthier and Finlay, Journal of Biological Chemistry 278(28): 25273-25276 (2003); Tampakaki et al., Cellular Microbiology 6(9): 805-816 (2004)). This system is highly specialized for the injection of specific proteins, known as effector proteins, directly into eukaryotic host cells. Once the effector proteins have been delivered to the host cell cytoplasm they mainly function in the disruption of host cell signaling and defense mechanisms. While the specific effector proteins made by individual bacterial species are different, the major components of the type III protein injection machinery are conserved among Gram-negative pathogens. In addition, the TTSS is homologous to the bacterial flagellum and many of the components are thought to be structurally and functionally similar (see, e.g., Piano and Ferracci, Molecular Microbiology 40(2): 284-293 (2001)). Like the type II secretion system, the type III system consists of several different proteins that appear to interact with one another via multiple protein-protein interactions.

Mutation or deletion of the genes encoding components of the TTSS has resulted in attenuation of disease in several animal models of infection, thus underlining the crucial role this system plays in pathogen virulence (see, e.g., Abe et al., Journal of Experimental Medicine 188(10): 1907-1916 (1998); Holder and Frank, Burns 27(2): 129-130 (2001); Stevens et al., Microbiology 150: 2669-2676 (2004); Smith and Lory, Infection and Immunity 72(3): 1677-1684 (2004); Warawa, FEMS Microbiology Letters 242(1): 101-108 (2005)). Moreover, damage due to lung infection by Pseudomonas aeruginosa can be ameliorated by immunization with antibodies to the Pseudomonas V-antigen or PcrV, a component of the TTSS that is involved in translocation of effector proteins (see, e.g., Sawa et al., Nature Medicine 5(4): 392-398 (1999); Shime et al., Journal of Immunology 167(10): 5880-5885 (2001). Additionally, treatment of burned mice with anti-PcrV antibodies has been shown to have a protective effect against subsequent infection by P. aeruginosa (see, e.g., Neely et al., Burns 31: 153-158 (2005)). Most importantly, the TTSS appears to play a crucial role in infections of humans since it has been shown that the presence of TTSS-expressing strains in ventilator-associated pneumonia is associated with poor clinic outcomes (see, e.g., Hauser, Critical Care Medicine 30(3): 521-528 (2002)) and patient strain isolates expressing exoU, a TTSS effector, are often highly virulent (see, e.g., Schulert et al., Journal of Infectious Diseases 188: 1695-1706 (2003)). Thus, the ability to inhibit the translocation and function of TTSS effector proteins would be beneficial for disease prevention. Accordingly, in some embodiments, the present invention provides compositions and methods for inhibiting translocation and function of TTSS effector proteins.

The translocation of proteins across cell membranes is an energetic process. For both the type II and type III secretion systems, the source of energy is thought to be derived from ATP hydrolysis as both systems have a component that shows similarity to a family of proteins that bind and hydrolyze ATP (see, e.g., Sandkvist, Molecular Microbiology, 40(2): 271-283 (2001); Sandkvist, Infection and Immunity, 69(6): 3523-3535 (2001); Tampakaki et al., Cellular Microbiology 6(9): 805-816 (2004)).

The most highly conserved region of these proteins is a Walker box A motif, which is responsible for nucleotide recognition and binding (see, e.g., Walker, EMBO Journal 1(8): 945-951 (1982)). Evidence suggests that these putative ATPases are oligomeric in nature and they appear to be cytoplasmic proteins that associate with the inner membrane where they can interact with other components of the secretion machinery (see, e.g., Ball et al., Journal of Bacteriology 181(2): 382-388 (1999); Pozidis et al., Journal of Biological Chemistry 278(28): 25816-25824 (2003); Camberg, Journal of_Bacteriology 187(1): 249-256 (2005)). In many cases, it has been demonstrated that these proteins bind and hydrolyze ATP in vitro (see, e.g., Eichelberg et al., Journal of Bacteriology 176(15): 4501-4510 (1994); Pozidis et al., Journal of Biological Chemistry 278(28): 25816-25824 (2003); Possot, Molecular Microbiology 12(2): 287-299 (1994); Pozidis et al., Journal of Biological Chemistry 278(28): 25816-25824 (2003); Akeda et al., Journal of Bacteriology 186(8): 2402-2412 (2004); Camberg, Journal of_Bacteriology 187(1): 249-256 (2005)) and an intact Walker box A motif has been shown to be essential for secretion in several organisms (see, e.g., Possot, Molecular Microbiology 12(2): 287-299 (1994); Pozidis et al., Journal of Biological Chemistry 278(28): 25816-25824 (2003); Akeda et al., Journal of Bacteriology 186(8): 2402-2412 (2004)). Furthermore, amino acid substitutions within the Walker box A motif, resulting in a secretion minus phenotype, are transdominant (see, e.g., Possot, Molecular Microbiology 12(2): 287-299 (1994); Akeda et al., Journal of Bacteriology 186(8): 2402-2412 (2004), Turner and Lowry, Molecular Microbiology 26(5): 877-887 (1997); Turner et al., Journal of Bacteriology 175(16): 4962-4969 (1994)).

Thus, in some embodiments, the present invention provides donor cells (e.g., pathogenic or non-pathogenic bacteria) comprising a transmissible plasmid, wherein the plasmid may be transferred (e.g., through conjugation) from the donor cell to a recipient cell (e.g., a pathogenic microorganism), resulting in the plasmid expressing its genetic material in the host. In some embodiments, the plasmid contains nucleic acid sequence that encodes dominant negative forms of proteins involved in type II or type III secretion systems (see, e.g., Example 1). In some embodiments, treating a subject exposed to a population of pathogenic microorganisms with the compositions and methods of the present invention attenuates the virulence of the recipient cells (see, e.g., Examples 3-5).

While an understanding of the mechanism is not necessary to practice the present invention and while the present invention is not limited to any particular mechanism of action, it is contemplated that, in some embodiments, the dominant negative effect derives from the oligomeric nature of type II or type III proteins (e.g., ATPase proteins), where mutant subunits assemble with their wild-type counterparts to form non-productive heterooligomers. In addition, in some embodiments, dominant negative ATPases identified herein form protein-protein interactions with other components of the secretion machinery, which, in some embodiments (e.g., when expressing dominant negative forms of ATPase components) block protein secretion (see, e.g., Sandkvist et al., EMBO Journal 14(8): 1664-1673 (1995); Kagami et al., Molecular Microbiology 27(1): 221-233 (1998); Ball et al., Journal of Bacteriology 181(2): 382-388 (1999); Sandkvist et al., Journal of Bacteriology 182(3): 742-748 (2000); Possot et al., Journal of Bacteriology 182(8): 2142-2152 (2000)).

As previously discussed, infections caused by pan-resistant strains of bacteria can be extremely difficult to treat with the current arsenal of antimicrobial agents and alternative agents are badly needed. Thus, there is a need for methods of treating pathogenic microorganisms. In some embodiments, the present invention provides treatments that target the pathways involved in virulence factor production, so as to disrupt or abate the production of these factors. In other embodiments, treatments are directed at interfering with the mechanisms of action or secretion of such virulence factors.

Accordingly, in some embodiments, the present invention provides conjugation to deliver genes comprising sequences encoding dominant negative proteins (e.g., effector proteins, or proteins involved in type II or type III secretion system) to a population of pathogenic microorganisms. In some embodiments, dominant negative effector proteins are selected from dominant negative forms of the group of proteins including, but not limited to, accessory cholera enterotoxin, adenylate cyclase toxin, adhesin, aerolysin toxin, aggregation substance, i.e., asa373, Agr A,B,C,D, SigB etc, alkaline protease, alpha toxin, alpha-haemolysin, alveolysin, anthrax toxin, APX toxin, beta toxin, botulinum toxin, bundle forming pilus structural subunit, C2 toxin, C3 toxin, C5A peptidase, cardiotoxin, chemotaxis, cholera toxin, ciliotoxin, clostridial cytotoxin, clostridial neutotoxin, collagen adhesion gene, crystal endotoxin, cyaA toxin, cytolysin, delta toxin, delta toxin, delta-lysin, diphtheria toxin, emetic toxin, endotoxin, staphylococcal enterotoxins A, B, C1, C2, C3, D, E, G, enterotoxin, exfoliative toxin, exotoxin, exotoxin A, exotoxin B, exotoxin C, extracellular elastase, fibrinogen, fibronectin binding protein, i.e., ffibA, filamentous hemagglutinin, fimbriae, gamma hemolysin, gelatinase, i.e., gelE, haemolysin, hemolysin B, hemagglutinin, histolyticolysin, IGG binding protein A, i.e., spaI, intimin, invasin, iron siderophores, ivanolysin, ivanolysin O, lantibiotic modifying enzyme, lantibiotic structural protein, lecithinase, ler (positive regulator of LEE genes), leukotoxin, lipoprotein signal peptidase, listeriolysin O, M protein, motility, neurotoxin, nonfimbrial adhesins, oedema factor, perfringolysin O, permease, pertussis toxin, phospholipase, pili, plasmid encoded regulator per, pneumolysin, poly-D-glutamic acid capsule, pore-forming toxin, proline permease, RNAIII, RTX toxin, serine protease, shiga toxin, siderophore/iron acquisition protein, SigA proteases, Spe A, Spe B, Spe C, Sta toxin, Stb toxin, streptolysin O, streptolysin S, superantigen, superoxide dismutase, TCP, tetanus toxin, thiol-activated cytolysin, tracheal cytotoxin, and TSST toxin (TSST-1).

In some embodiments, the present invention provides a method of blocking a secretion pathway (e.g., type II, type III, and type IV secretion system pathways). In some embodiments, blocking of a secretion pathway attenuates pathogen virulence by disrupting protein secretion through effector secretion systems (i.e. type II, type III, or type IV secretion system pathways).

In some embodiments, the present invention provides compositions and methods for altering protein production (e.g., production of virulence factors, effector proteins, secretion machinery proteins, etc.). In some embodiments, the present invention provides compositions and methods for inhibiting translocation and function of TTSS effector proteins.

For example, in some embodiments, the present invention provides compositions and methods for exploiting the dominant negative (DN) nature of mutations such as Walker box mutations (see, e.g., Example 1). In some embodiments, expression of DN Walker box mutations results in a reduction or blocking of protein secretion (e.g., virulence factor secretion from type II or type III secretion pathways), thereby attenuating virulence and/or pathogenicity of the microorganism. For example, in some embodiments, the present invention provides DN mutations in the putative ATPases of the type II and type III secretion systems and methods of expressing the DN mutants in trans in a population of pathogenic bacteria (e.g., strain PA14) (see, e.g., Examples 4-6, demonstrating that expression using compositions and methods of the present invention, in trans, of the DN alleles attenuate pathogenicity in a murine pneumonia model). It is contemplated that expression of DN mutants of ATPases as a method for pathogen attenuation is broadly applicable to all microorganisms (e.g., Gram-negative pathogens) due to the conserved nature of core components of these secretion systems and their energy requirements).

Thus, in some embodiments, the present invention provides DN mutations of ATPases and their expression in trans in populations of a genus of bacteria, selected from the group consisting of Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium, Treponema, Borrelia, Leptospira, and Chlamydiae, in order to attenuate, among other things, virulence, growth, and/or pathogenicity.

It is contemplated that alteration of recipient cells according to the present invention also comprises altering such cells so as to alter the response of such recipient cells to drugs, e.g., antibiotics. It is contemplated that any alteration to a recipient cell to alter that recipient cell's metabolism such that said recipient cell becomes susceptible to, or has increased susceptibility or response to a drug is encompassed by the methods, compositions and systems of the present invention. In some embodiments, a transmissible plasmid of the present invention encodes a factor capable of inhibiting a pathogen's ability to destroy or inactivate a drug such as an antibiotic. For example, an expression product of a transmitted plasmid may disrupt the ability of a pathogen enzyme capable of destroying or inactivating an antibiotic. In other embodiments, an expression product of a transmitted plasmid may provide a receptor for an antibiotic on or in the pathogen cell, or restore a defective receptor for an antibiotic on or in the pathogen cell. In yet other embodiments, an expression product of a transmitted plasmid may facilitate entry of an antibiotic into the pathogen cell, or inhibit the pathogen cell's ability to transport the antibiotic out of the pathogen cell.

In some embodiments, an expression product of a transmitted plasmid may serve to metabolize an inactive drug such as a prodrug into an active form, e.g., a form to which the recipient cell is responsive. The use of prodrugs that are metabolized to form an active drug can be particularly beneficial in bypassing drug resistance mechanisms, and in providing selective treatment, e.g., targeting cells that have received an appropriate transmissible plasmid.

Disruption of Secretion Machinery

There is genetic and biochemical evidence for multiple protein-protein interactions between the different components of the secretion systems (see, e.g., Sandkvist et al., Journal of Bacteriology 182, 742-748 (2000); Possot et al., Journal of Bacteriology 182, 2142-2152 (2000); Sandkvist et al., EMBO Journal 14, 1664-1673 (1995); Kagami, et al., Molecular Microbiology 27, 221-233 (1998)). The secretion systems are elaborate, sophisticated machines with multiple interacting components. Thus, it is contemplated that, in some embodiments, any identified mutant that disrupts such interactions can be used to disrupt assembly and/or function of the secretion systems (e.g., type II or type II systems).

For example, experiments conducted using the compositions and methods of the present invention utilized dominant negative (DN) mutants of the type II or type III protein secretion systems of Gram negative bacteria. DN mutations were made in the Walker A box motif of the protein subunit that provides the energy for translocation (i.e. an ATPase) (see, e.g., Example 2).

Thus, in some embodiments, nucleic acid sequences encoding proteins (e.g., dominant negative proteins) are encoded on a plasmid (e.g., RK2, R6K, pCUI, p15A, pIP501, pAM1, pCRG1600 or pCON4-78) and placed into a donor cell (e.g., a pathogenic or non-pathogenic genus of bacteria) that posses the ability to conjugatively transfer the plasmid to a recipient cell (e.g., a pathogenic or non-pathogenic genus of bacteria) for expression of the protein. In addition to those already described, exemplary plasmids and donor cells that find use in the present invention include, but are not limited to, those of U.S. Pat. App. Nos. 20040137002, 20040224340, and 10/884,257, herein incorporated by reference in their entireties for all purposes. In some embodiments, the plasmid used is self-transmissible. While an understanding of the mechanism is not necessary to practice the present invention and while the present invention is not limited to any particular mechanism of action, it is contemplated that, in some embodiments, expression of dominant negative proteins in trans results in “subunit poisoning” of the wild-type protein secretion machinery (e.g., by disruption of the functioning of the ATPase or by disrupting protein-protein interactions between the ATPase and other components of the machinery).

In some embodiments, compositions and methods of the present invention are used to disrupt protein-protein interactions within the type III machinery (see, e.g., Jouihri et al., Molecular Microbiology 49, 755-767 (2003). Schuch and Maurelli, Journal of Bacteriology 183, 6991-6998 (2001); Jackson et al., FEMS Microbiology Letters 186, 85-90 (2000). Creasey et al., Microbiology 149, 2093-2106 (2003); Alegria et al., Journal of Bacteriology 186, 6186-6197 (2004)). In some embodiments, compositions and methods of the present invention are used to disrupt protein-protein interactions within the type IV machinery (see, e.g., Alegria et al., Journal of Bacteriology 187, 2315-2325 (2005); Krall et al., PNAS 99, 11405-11410 (2002). Atmakuri et al., Molecular Microbiology 54, 1199-1211 (2004); and Shamaei-Tousi et al., Journal of Bacteriology 186, 4796-7801 (2004)).

Disruption of Gene Expression

A review of the literature pertaining to bacterial protein secretion systems (e.g., types I-IV) indicates that there are multiple layers of regulation of the systems (e.g., gene and protein expression, secretion, etc.), providing a number of opportunities to disrupt these systems. Thus, in addition to the above described approaches (e.g., using dominant negative mutations to prevent secretion of proteins—effector proteins—from Gram-negative bacteria), the present invention provides alternative ways to attenuate virulence in unwanted pathogens. For example, in some embodiments, the present invention provides compositions and methods for attenuating pathogenicity via altering (e.g., inhibiting) protein-protein interactions necessary for expression of genes (e.g., genes encoding proteins that function in the secretory pathway, effector proteins, homeostasis proteins, etc.).

For example, the genes for the type III secretion and translocation machinery in P. aeruginosa are encoded in four operons that are positively regulated by a central transcriptional regulator, ExsA (see, e.g., Hovey and Frank, Journal of Bacteriology 177, 4427-4436 (1995)). Additionally, there is evidence for a negative feedback loop that appears to be controlled by an anti-activator, ExsD, and genetic evidence suggests that negative inhibition is due to interactions between ExsA and ExsD (NcCaw et al., Molecular Microbiology 46, 1123-1133 (2002)). There is also now evidence for a third regulator, ExsC, which appears to antagonize the activity of ExsD (i.e. anti-anti-activator) and biochemical and genetic evidence show that ExsD and ExsC interact (Dasgupta et al., Molecular Microbiology 53, 297-308 (2004)). A model has been proposed whereby an interaction between a hypothetical type III effector protein (i.e. one of the proteins that are “injected” into host cells) and ExsC determines whether ExsD will be free to interact with ExsA and thus, prevent transcription of type III genes (Dasgupta et al., Molecular Microbiology 53, 297-308 (2004)).

Since there are multiple protein-protein interactions occurring in this regulatory mechanism, it is contemplated that, in some embodiments, compositions and methods of the present invention are used to disrupt one or more of these interactions thereby disrupting the type III secretion system (e.g., blocking secretion of proteins). For example, the central transcriptional regulator (ExsA) of this system is a member of the bacteial AraC family of proteins, which have been well studied. Many different pathogens have similar AraC proteins, including, but not limited to, Pseudomonas, Shigella flexneri, Yersinia spp., Salmonella typhimurium, and enteropathogenic E. coli, each of which play important roles in type III secretion (Francis et al., Current Opinion in Microbiology 5, 166-172 (2002)).

Thus, in some embodiments, compositions and methods of the present invention inhibit, in trans, interaction between EsxA (or equivalent proteins thereof found in other bacteria) and other proteins involved in gene expression. The identification of specific amino acid residues that are responsible for the various protein-protein interactions between proteins, as well as the dissection of protein-protein interactions, are routinely carried out under ordinary conditions by those skilled in the art using well known genetic and biochemical techniques.

The regulatory scheme presented above is a variation on the transcriptional regulation of the well-studied flagellar assembly system, which is considered to be the prototype of the type III secretion system (see, e.g., Aldridge and Hughes, Current Opinion in Microbiology 5, 160-165 (2002)). The type III secretion systems generally have a negative feedback loop, whereby the absence of a functional secretion system in the cell results in a lack of component gene expression. In the flagellar system, transcription of component genes is positively regulated by a σ-factor, FliA. σ-factor is a component of RNA polymerase holoenzyme in bacteria, which assists RNA polymerase to recognize a promoter sequence (Travers and Burgess, Nature 222, 537-40 (1969)). FliA is a σ²⁸-factor facilitating gene expression of sets of flagellar components. FliA binds tightly to the anti-a factor FlgM, and the function of FliA is inhibited by this protein-protein interaction. Interestingly, the anti-σ factor FlgM is a substrate for secretion through the flagellar assembly system, hook-basal body (Chilcott and Hughes, Microbiol Mol Biol Rev 64, 694-708 (2000)). Before completion of the flagellar assembly, FlgM is actively secreted outside of the cell, thus freeing up FliA for transcription activation. Once the flagella is completely assembled, FlgM is no longer secreted, and accumulates within the cell. The increased amount of FlgM within the cell leads the FliA-FlgM interaction, shutting down the synthesis of flagellar components. Working on similar principles, in some embodiments, the present invention provides a gene encoding an anti-sigma factor that is delivered to a pathogen through conjugation, where its expression inhibits expression of genes essential for the type III secretion system.

Many protein components serve essential roles in the secretion through type II, type III and type IV secretion systems. Thus, in some embodiments, one or more of these key components are targeted via the compositions and methods of the present invention (e.g., via donor cells, carrying transmissible plasmids, capable of conjugative transfer of the plasmids to recipient cells, the subsequent expression of genetic material encoded upon the plasmids in the recipient cells, and disruption of these systems in the recipient cells). Since a number of genes can be mobilized through conjugation from a donor bacterium to a recipient bacterium (e.g., a pathogen) it is contemplated that the compositions and methods of the present invention permit inhibition of multiple levels of secretion systems simultaneously (e.g., concurrent inhibition of transcription and “subunit poisoning”). Furthermore, the compositions and methods of the present invention (e.g., the conjugative delivery system) permit the expression of any protein component associated with virulence in a target pathogen (e.g., disrupting specific protein-protein interactions to reduce its virulence). Thus, it is contemplated that compositions and methods of the present invention can be used to alter pathogen virulence (e.g., through blocking type II or type III secretion systems) and growth (e.g., through inhibiting gene expression required for bacterial cell growth).

Therapeutics

The compositions and methods of the present invention find utility for treatment of humans and in a variety of veterinary, agronomic, horticultural and food processing applications.

For human and veterinary use, and depending on the cell population or tissue targeted for protection (e.g., via killing of pathogenic target cells), the following modes of administration of the compositions (e.g., donor bacterial cells comprising a transmissible plasmid) of the present invention are contemplated: topical, oral, nasal, pulmonary/bronchial (e.g., via an inhaler), ophthalmic, rectal, urogenital, subcutaneous, intraperitoneal and intravenous. The bacteria preferably are supplied as a pharmaceutical preparation, in a delivery vehicle suitable for the mode of administration selected for the patient being treated. The term “patient” or “subject” as used herein refers to humans or animals (animals being particularly useful as models for clinical efficacy of a particular donor strain).

For instance, to deliver the donor bacterial cells to the gastrointestinal tract or to the nasal passages, the preferred mode of administration is by oral ingestion or nasal aerosol, or by feeding (alone or incorporated into the subject's feed or food). In this regard, it should be noted that probiotic bacteria, such as Lactobacillus acidophilus, are sold as gel capsules containing a lyophilized mixture of bacterial cells and a solid support such as mannitol. When the gel capsule is ingested with liquid, the lyophilized cells are re-hydrated and become viable, colonogenic bacteria. Thus, in a similar fashion, donor bacterial cells of the present invention can be supplied as a powdered, lyophilized preparation in a gel capsule, or in bulk for sprinkling into food or beverages. The re-hydrated, viable bacterial cells will then populate and/or colonize sites throughout the upper and lower gastrointestinal system, and thereafter come into contact with the target pathogenic bacteria.

For topical applications, the bacteria may be formulated as an ointment or cream to be spread on the affected skin surface. Ointment or cream formulations are also suitable for rectal or vaginal delivery, along with other standard formulations, such as suppositories. The appropriate formulations for topical, vaginal or rectal administration are well known to medicinal chemists.

The present invention will be of particular utility for topical or mucosal administrations to treat a variety of bacterial infections or bacterially related undesirable conditions. Some representative examples of these uses include, but are not limited to, treatment of (1) conjunctivitis, caused by Haemophilus sp., and corneal ulcers, caused by Pseudomonas aeruginosa; (2) otititis extema, caused by Pseudomonas aeruginosa; (3) chronic sinusitis, caused by many Gram-positive cocci and Gram-negative rods, and for general decontamination of bronchii; (4) cystic fibrosis, associated with Pseudomonas aeruginosa; (5) enteritis, caused by Helicobacterpylori (ulcers), Escherichia coli, Salmonella typhimurium, Canipylobacter and Shigella sp.; (6) open WO 02/18605 PCTIUSOI/27028 associated with Gardnerella vaginalis and other anaerobes; and (12) gingivitis and/or tooth decay caused by various organisms.

In other embodiments, the compositions and methods of the present invention find application in the treatment of surfaces for the attenuation or growth inhibition of unwanted bacteria (e.g., pathogens). For example, surfaces that may be used in invasive treatments such as surgery, catheterization and the like may be treated to prevent infection of a subject by bacterial contaminants on the surface. It is contemplated that the methods and compositions of the present invention may be used to treat numerous surfaces, objects, materials and the like (e.g., medical or first aid equipment, nursery and kitchen equipment and surfaces) to control bacterial contamination thereon.

In other embodiments, the compositions may be impregnated into absorptive materials, such as sutures, bandages, and gauze, or coated onto the surface of solid phase materials, such as surgical staples, zippers and catheters to deliver the compositions to a site for the prevention of microbial infection. Other delivery systems of this type will be readily apparent to those skilled in the art.

Pharmaceutical preparations comprising the donor bacteria are formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of the donor bacteria cells calculated to produce the desired antibacterial (e.g., attenuation of pathogenicity) effect in association with the selected pharmaceutical carrier. Procedures for determining the appropriate dosage unit are well known to those skilled in the art.

Dosage units may be proportionately increased or decreased based on the weight of the patient. Appropriate concentrations for achieving eradication of pathogenic bacteria in a target cell population or tissue may be determined by dosage concentration curve calculations, as known in the art.

Other uses for the donor cells of the invention are also contemplated. These include a variety of agricultural, horticultural, environmental and food processing applications. For example, in agriculture and horticulture, various plant pathogenic bacteria may be targeted in order to minimize plant disease. One example of a plant pathogen suitable for targeting is Erwinia amylovora, the causal agent of fire blight.

Similar strategies may be utilized to reduce or prevent wilting of cut flowers. In veterinary or animal agriculture, the compositions (e.g., plasmid systems) of the invention may be incorporated into animal feed (chicken, cattle) to reduce bio-burden or to attenuate certain pathogenic organisms (e.g., Salmonella). In other embodiments, the invention may be utilized on meat or other foods to attenuate or neutralize pathogenic bacteria (e.g., E. coli 0157:H7 on meat).

Environmental utilities comprise, for example, engineering Bacillus thurengiensis and one of its conjugative plasmids to deliver and conditionally express insecticidal agents (e.g., for the control of mosquitoes that disseminate malaria or West Nile virus). In such applications, as well as in the agricultural and horticultural applications described above, formulation of the plasmids and donor bacteria as solutions, aerosols, or gel capsules are contemplated.

In preferred embodiments of the present invention, certain features are employed in the plasmids and donor cells of the invention to minimize potential risks associated with the use of DNA or genetically modified organisms in the environment. For instance, in environmentally sensitive circumstances it may be preferable to utilize non-self-transmissible plasmids. Thus, in some embodiments, the plasmids are mobilizable by conjugative machinery but are not self-transmissible. As discussed herein, this may be accomplished in some embodiments by integrating into the host chromosome all tra genes whose products are necessary for the assembly of conjugative machinery. In such embodiments, plasmids are configured to possess only an origin of transfer (oriT). This feature prevents the recipient, before or even after it dies, from transferring the plasmid further.

Another biosafety feature comprises utilizing conjugation systems with predetermined host-ranges. As discussed above, certain elements are known to function only in few related bacteria (narrow-host-range) and others are known to function in many unrelated bacteria (broad-host-range or promiscuous) (del Solar et al., Mol. Microbiol. 32: 661-666, (1996); Zatyka and Thomas, FEMS Microbiol. Rev. 21: 29 1 319, (1998)). Also, many of those conjugation systems can function in either Gram-positive or Gram-negative bacteria but generally not in both (del Solar, 1996, supra; Zatyka and Thomas, 1998, supra).

Inadvertent proliferation of antibiotic resistance is minimized in this invention by avoiding the use of antibiotic resistance markers. In a preferred alternative approach, the gene responsible for the synthesis of an amino acid (i.e. serine) can be mutated, generating the requirement for this amino acid in the donor. Such mutant bacteria will prosper on media lacking serine provided that they contain a plasmid with the ser gene whose product is needed for growth.

Thus, the invention contemplates the advantageous use of plasmids containing the Ser gene or one of many other nutritional genetic markers. These markers permit selection and maintenance of the plasmids in donor cells.

Another approach comprises the use of restriction-modification systems to modulate the host range of plasmids. Conjugation and plasmid establishment are expected to occur more frequently between taxonomically related species in which plasmid can evade restriction systems and replicate. Type II restriction endonucleases make a double-strand break within or near a specific recognition sequence of duplex DNA. Cognate modification enzymes can methylate the same sequence and protect it from cleavage. Restriction-modification systems (RM) are ubiquitous in bacteria and archaebacteria but are absent in eukaryotes. Some of RM systems are plasmid-encoded, while others are on the bacterial chromosome (Roberts and Macelis, Nucl. Acids Res. 24: 223-235, (1998)). Restriction enzymes cleave foreign DNA such as viral or plasmid DNA when this DNA has not been modified by the appropriate modification enzyme. In this way, cells are protected from invasion of foreign DNA. Thus, by using a donor strain producing one or more methylases, cleavage by one or more restriction enzymes could be evaded. Site directed mutagenesis is used to produce plasmid DNA that is either devoid of specific restriction sites or that comprises new sites, protecting or making plasmid DNA vulnerable, respectively against endonucleases. Broad-host range plasmids (e.g. RN) may evade restriction systems simply by not having many of the restriction cleavage sites that are typically present on narrow-host plasmids (Willkins et al., J. Mol. Biol 258, 447-456 (1996)).

In some embodiments, the present invention utilizes environmentally safe bacteria as donors. Safe bacteria are known in the art. For example, delivery of DNA vaccines by attenuated intracellular Gram-positive and Gram-negative bacteria has been reported (Dietrich et al., 2001 Vaccine 19, 2506-2512; Grillot-Courvalin et al., 1999 Current Opinion in Biotech. 10, 477-481). In addition, the donor strain can be one of thousands of harmless bacteria that colonize the non-sterile parts of the body (e.g., skin, gastrointestinal, urogenital, mouth, nasal passages, throat and upper airway systems).

In another strategy, non-viable donors are utilized instead of living cells. For example, minicells and maxicells are well studied model systems of metabolically active but nonviable bacterial cells. Minicells lack chromosomal DNA and are generated by special mutant cells that undergo cell division without DNA replication. If the cell contains a multicopy plasmid, many of the minicells will contain plasmids. Minicells neither divide nor grow. However, minicells that possess conjugative plasmids are capable of conjugal replication and transfer of plasmid DNA to living recipient cells. (see, e.g., U.S. Pat. No. 4,968,619).

Maxicells are cells that are treated so as to destroy their chromosomal DNA, while retaining the function of plasmids that they contain. Maxicells can be obtained from a strain of E. coli that carries mutations in the key DNA repair pathways (recA, uvrA and phr). Because maxicells lack so many DNA repair functions, they die upon exposure to low doses of UV. Importantly, plasmid molecules (e.g., pBR322) that do not receive UV irradiation continue to replicate. Transcription and translation (plasmid-directed) can occur efficiently under such conditions (Sancar et al., J. Bacteriol. 137: 692-693 (1979)), and the proteins made prior to irradiation should be sufficient to sustain conjugation. This is supported by the following two observations: i) that streptomycin-killed cells remain active donors, and ii) that transfer of conjugative plasmids can occur in the presence of antibiotics that prevent de novo gene expression (see, e.g., Heineman and Ankenbauer, J. Bacteriol. 175. 583-588 (1993); Cooper and Heineman, Plasmid 43, 171-175 (2000)). Accordingly, UV-treated maxicells will be able to transfer plasmid DNA to live recipients. It should also be noted that the conservation of recA and uvrA genes among bacteria should allow maxicells of donor strains other than E. coli to be obtained.

In some embodiments, the present invention utilizes non-dividing cells (e.g., a described in U.S. patent application Ser. No. 10/884,257, filed Jul. 2, 2004, incorporated herein by reference in its entirety for all purposes) as donor cells. Non-dividing cells are generally treated such that the ability to divide and grow is removed but conjugation efficiency is preserved. In preferred embodiments, non-dividing cells are treated such that chromosomal DNA is damaged but is not destroyed to the same extent as it is in the creation of maxicells.

In some embodiments, modified microorganisms that cannot function because they contain temperature-sensitive mutation(s) in genes that encode for essential cellular functions (e.g., cell wall, protein synthesis, RNA synthesis, as described, for example, in U.S. Pat. No. 4,968,619) are used.

For many approaches, conditionally replicating plasmids can be used. Such plasmids, can replicate in the donor but cannot replicate in the recipient bacterium simply because their cognate replication initiator protein (e.g., Rep) is produced in the former cells but not the latter cells. In some embodiments, a variant plasmid contains a temperature-sensitive mutation in the rep gene, so it can replicate only at temperatures below 37 C. Hence, its replication will occur in bacteria applied on skin but it will not occur if such bacteria break into the body's core.

As used herein, the terms “donor bacterial cell” and “donor cell” refer to any of the above-listed cells, including dividing and non-dividing bacterial cells (e.g., minicells and maxicells), as well as conditionally non-functional cells.

The donor bacteria of the present invention can be applied to skin (e.g., burned or infected skin) as a therapeutic or applied as a prophylactic to prevent bacterial infection. It is contemplated that the donor cells can be applied to the skin surface via a number of delivery mechanisms.

For example, the compositions (e.g., donor bacteria comprising killer plasmids, or plasmids configured to otherwise attenuate a pathogen) of the present invention can be applied (e.g., to a skin burn or wound surface) by multiple methods, including, but not limited to: being suspended in a solution (e.g., colloidal solution) and applied to a surface; being suspended in a solution and sprayed onto a surface using a spray applicator; being mixed with fibrin glue and applied (e.g., sprayed) onto a surface (e.g., skin burn or wound); being impregnated onto a wound dressing or bandage and applying the bandage to a surface (e.g., an infection or wound); being applied by a controlled-release mechanism; being impregnated on one or both sides of an acellular biological matrix that can then be placed on a surface (e.g., skin wound or burn) thereby protecting at both the wound and graft interfaces; being applied as a liposome; or being applied on a polymer.

While an understanding of the mechanism is not necessary to practice the present invention and while the present invention is not limited to any particular mechanism of action, it is contemplated that, in some embodiments, once on the skin or wound surface, donor bacteria come into contact with the targeted pathogenic bacteria and pass antibacterial genes via the conjugation process into the targeted pathogens, killing the pathogens.

In preferred embodiments, a wound remains free of infection due to pathogenic bacteria (i.e., recipients/targets) contacting donor bacteria and being killed. Donor bacteria can be any strain of bacteria including any Gram negative or Gram positive bacterium. For example, in some embodiments, the present invention provides E. coli, Pseudomonas sp., Klebsiella sp., Enterobacter sp., Acinetobacter sp., Lactobacillus sp., Lactococcus sp., Staphyloccus sp., Streptococcus sp., Enterococcus sp., or Bacteroides sp. as donor bacteria.

### EXPERIMENTAL

The following examples are provided in order to demonstrate and further illustrate certain preferred embodiments and aspects of the present invention and are not to be construed as limiting the scope thereof.

In the experimental disclosure that follows, the following abbreviations apply: ° C. (degrees Centigrade); cm (centimeters); g (grams); 1 or L (liters); μg (micrograms); μl (microliters); μm (micrometers); μM (micromolar); μmol (micromoles); mg (milligrams); ml (milliliters); mm (millimeters); mM (millimolar); mmol (millimoles); M (molar); mol (moles); ng (nanograms); nm (nanometers); nmol (nanomoles); N (normal); pmol (picomoles); bp (base pairs); cfu (colony forming units); Invitrogen (Invitrogen, Carlsbad, Calif.); lacOP (region encoding the E. coli lac operator/promoter); xcpR G268A (gene encoding P. aeruginosa type II secretion system ATPase, G268A dominant negative allele); pscN K176G (gene encoding the P. aeruginosa type III secretion system ATPase, K176G DN allele); Kan (determinant for kanamycin resistance); Cm (determinant for chloramphenicol resistance); Tral (region encoding genes responsible for conjugative transfer); Control (region encoding control region); oriV (region encoding the origin of vegetative replication); oriT (region encoding the origin of conjugative transfer); TetR (gene encoding repressor of tetA); TetA (gene encoding resistance to tetracycline); Rep (region encoding genes responsible for replication); Primase (region encoding genes responsible for replication); Tra2 (region encoding genes responsible for conjugative transfer).

### EXAMPLE 1

### Materials and Methods

Bacterial Strains and Media

Vector construction utilized Escherichia coli strains JM109 (C. -Yanisch-Perron, J. Vieira, J. Messing, Gene 33, 103-19 (1985)) and TOP10 (Invitrogen). For testing of DN alleles in the murine pneumonia model, Pseudomonas aeruginosa PA14 was used as a pathogen, and the host strain for the constructed plasmids (L. G. Rahme et al., Science 268, 1899-902 (1995)). All cloning was performed using standard methods known in the art, and using Luria Bertani (LB) growth media supplemented with the appropriate drugs for selection of plasmids: carbenicillin, 100 μg/ml; kanamycin, 25 μg/ml; tetracycline, 15 μg/ml; chlormaphenicol, 20 μg/ml. For growth of P. aeruginosa, LB broth was supplemented with 50 μg/ml rifampicin and plasmids were selected by the addition of 50 μg/ml tetracycline.

Construction of Plasmids

Construction of pCON4-78, a self-transmissible cloning vector derived from RK2. RK2 is a self-transmissible conjugative plasmid with the size of 60,099 bp (W. Pansegrau et al., J Mol Biol 239, 623-63 (1994)). RK2 is one of the best studied conjugative plasmids, and functions of most of the genes encoded on the RK2 plasmid have been studied. RK2 possesses a robust conjugation efficiency, however, due to its size and lack of useful restriction sites, genetic manipulation of this molecule was inefficient. To solve this problem, a vector useful for general cloning was generated. To achieve this, a number of non-essential genes for conjugation were deleted from RK2 and convenient cloning sites were introduced. This process included elimination of KilA, KilE, Tn1, KilC, IS21, Par and Mrs regions, and introduction of unique cloning sites: HindIII, BclI, BamHI, PacI, EcoRI, XbaI, AscI, AsiSI, AseI, SacI, NheI, MluI and NsiI.

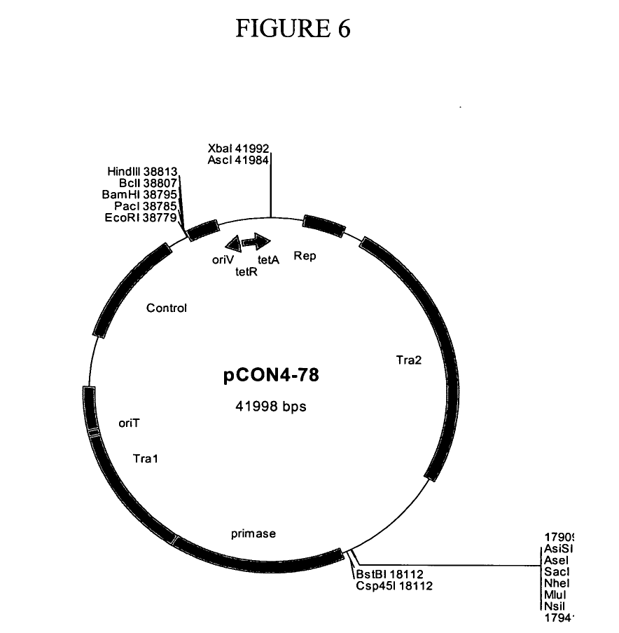

This plasmid construct was designated as pCON4-78 (see, FIG. 6). pCON4-78 conjugates as efficiently as its parental plasmid RK2. Since the size was significantly reduced from its parental plasmid (60 kb to 42 kb), handling of the plasmid became easier and more efficient. For the plasmid constructions, HindIII-EcoRI and SacI-MluI sites were used for cloning.

Construction of pCON22-98 Designed to Target the Type II Secretion System

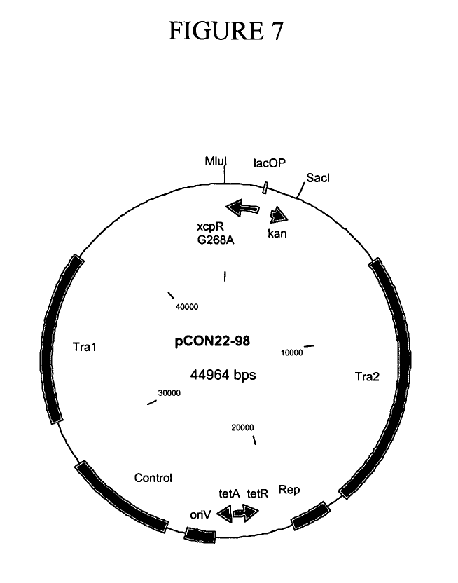

The gene, xcpR, encoding the ATPase subunit of the type II secretion system was cloned from P. aeruginosa PA14 using PCR, and was verified by DNA sequencing. The glycine (G) residue located at position 268 of this protein was changed to alanine (A) using site-directed mutagenesis using a kit purchased from Invitrogen. The modification of the sequence was verified by DNA sequencing and designated as xcpR G268A. The mutant xcpR was cloned downstream of the lacOP promoter and Shine-Dalgarno sequence for expression of the gene (J. Shine, L. Dalgarno, Eur J Biochem 57, 221-30 (1975)). The Shine-Dalgarno sequence is a ribosome biding site located adjacent to the translation initiation site for optimum protein synthesis. This expression cassette, lacOP-xcpR G268A was further subcloned into the SacI-MluI site of pCON4-78 to generate pCON22-98. The structure of pCON22-98 is depicted in FIG. 7 Prior to this subcloning, the expression cassette was combined with a kanamycin-resistance determinant to make this cloning process more efficient. The primase region exists as shown in pCON4-78, but it is not shown in the pCON22-98 plasmid map.

Construction of pCON22-35, Designed to Target the Type III Secretion System

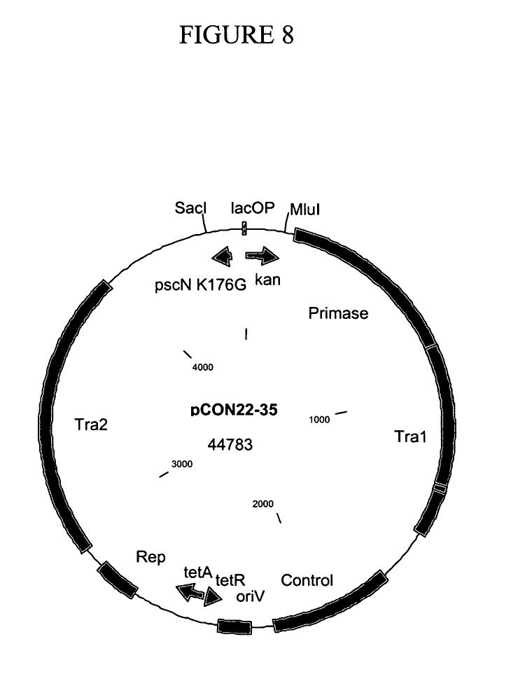

The gene, pscN, encoding the ATPase subunit of the type III secretion system was cloned from P. aeruginosa PA14 using PCR, and was verified by DNA sequencing. The lysine (K) residue located at position 176 of this protein was changed to glycine (G) using site-directed mutagenesis using a kit purchased by Invitrogen. The modification of the sequence was verified by DNA sequencing, and designated as pscN K176G. The mutagenized pscN was cloned downstream of the lacOP promoter and Shine-Dalgarno sequence for expression of the gene (J. Shine, L. Dalgarno, Eur J Biochem 57, 221-30 (1975)). This expression cassette, lacOP-pscN K176G was further subcloned into the SacI-MluI site of pCON4-78 to generate pCON22-35. The structure of pCON22-35 is depicted in FIG. 8. Prior to this subcloning, the expression cassette was combined with a kanamycin-resistance determinant to make this cloning process more efficient.

Construction of pCON22-62 Designed to Target both Type II and Type III Secretion Systems

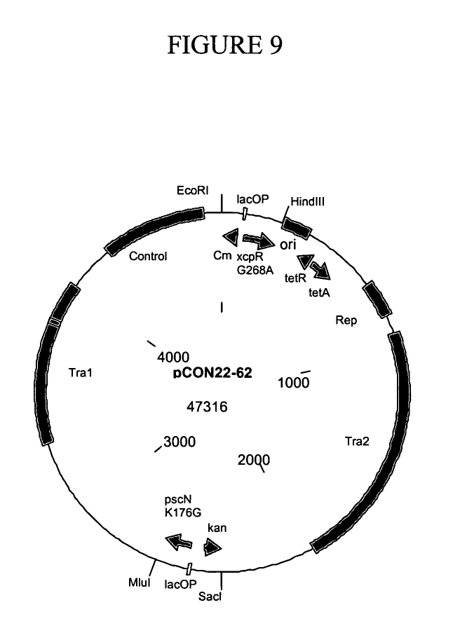

Both mutant alleles generated per the above description were combined onto a single plasmid to inhibit both the type II and the type III secretion systems. The expression cassette for the type II secretion system, lacOP-xcpR G268A, was combined with chloramphenicol-resistance determinant (Cm), and further cloned into the EcoRI and HindIII sites of pCON22-35 to generate pCON22-62 (see, FIG. 9). The Cm was used to select the correct recombinant plasmid. The structure of this plasmid is depicted below. The primase region exists as shown in pCON4-78, but it is not shown in the pCON22-62 plasmid map.

### EXAMPLE 2

### Monitoring Pathogen Virulence—Pseudmonas aeruginosa PA14

P. aeruginosa PA14 (L. G. Rahme et al., Science 268, 1899-902 (1995)) was chosen as a model target pathogen and used in a murine pneumonia model in order to test and demonstrate the utility of the invention. PA14 is virulent to number of organisms including plants, animals and worms. First, in order to demonstrate attenuation of virulence, normal levels of virulence for the pathogen in the murine pneumonia model were determined.

In this model, the death of an animal is an end point of the assay. Different amounts of the pathogen were nasally instilled into mice lungs, and the condition and health status of individual mice monitored for up to 7 days. It was expected that the LD₅₀ would fall somewhere around 3 days after administration of the pathogenic agent. Due to relatively large variations between experiments, it was difficult to obtain an exact LD50. However, the experiment did identify a dose, one not so large that it caused the immediate death of the mice, nor too small that it was not lethal within 7 days. The following doses and conditions were used:

| Number of mice | breed | pathogen | dose/cfu |
|---|---|---|---|
| 10 | Swiss Webster | PA14 | 10⁷ |
| 10 | Swiss Webster | PA14 | 10⁶ |
| 10 | Swiss Webster | PA14 | 10⁵ |
| 10 | Swiss Webster | PA14 | 10⁴ |

PA14 was grown overnight, and 4 different dilutions were made (1×10⁷, 1×10⁶, 1×10⁵ and 1×10⁴ cfu per animal), administered to the mice, and infected animals were monitored for survival. Bacteria were grown in LB medium supplemented with 50 μg/ml of rifampicin overnight at 37° C. The bacteria were harvested, resuspended in saline, and the cell density was adjusted based on OD₆₀₀. Appropriate amounts of cell suspensions were prepared by dilution. For instillation/administration, the animal was anesthetized with isoflurane, and the bacterial suspension was nasally instilled in a volume of 50 ul. When an animal became terminally sick, it was euthanized and considered to be killed by pneumonia/sepsis.

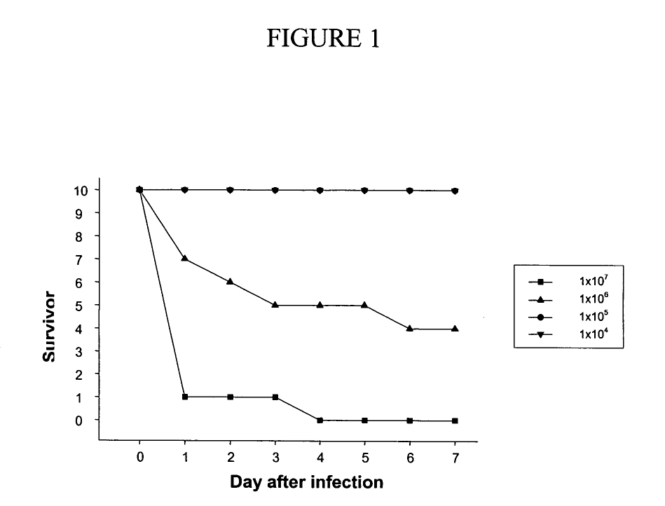

The results are shown in FIG. 1. Note the lowest two dilutions are overlapping in the graph. From these experiments, it was determined that 1×10⁷ cfu would be used as an initial dose for testing the methods and compositions of the present invention. Note, however, it is contemplated that exposure (e.g., of a subject) to any number of pathogenic organisms, whether the number of organisms is larger or small than the doses used herein, and use of the compositions and methods discussed herein, will lead to attenuation of the pathogenicity/virulence of the pathogens.

### EXAMPLE 3

### Effect of Plasmid Vector on Virulence

The RK2 system was used to deliver mutant genes among a population of P. aeruginosa. Thus, it is important to demonstrate that the plasmid itself does not have an effect on the virulence of the PA14 pathogen. In this experiment, the virulence of P. aeruginosa carrying and not carrying the RK2 plasmid was analyzed. Based on previous experiments (see, e.g., Example 2), doses of the pathogen starting at 1×10⁶ cfu per animal were chosen. The experimental methods were the same as those previously described (see, e.g., Example 2). For selection of RK2, the growth medium was supplemented with 50 μg/ml tetracycline.

The following doses and conditions were used:

| Number of mice | breed | pathogen | dose/cfu |
|---|---|---|---|
| 8 | Swiss Webster | PA14/RK2 | 10⁶ |
| 8 | Swiss Webster | PA14/RK2 | 10⁷ |
| 8 | Swiss Webster | PA14/RK2 | 10⁸ |
| 8 | Swiss Webster | PA14 | 10⁶ |
| 8 | Swiss Webster | PA14 | 10⁷ |

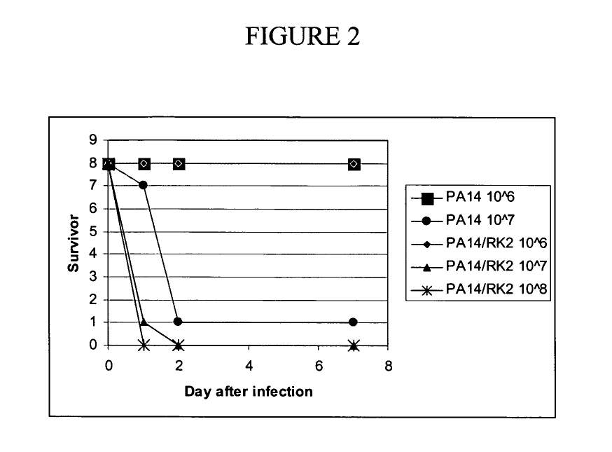

The results are shown in FIG. 2. As observed in Example 2, a dose of 10⁷ cfu of PA14 was lethal within two days. There was no difference in virulence between PA14 without RK2 and PA14 carrying RK2. Thus, the presence of the RK2 plasmid does not appear to significantly alter the virulence of PA14.

### EXAMPLE 4

### Virulence of Strains Containing Double Mutant Attenuator Plasmids

Two different types of toxin secretion systems, type II (T2) and type III (T3) secretion systems, were targeted in P. aeruginosa. In this example, a plasmid carrying both mutant genes (i.e., mutant pscN and mutant xcpR, termed T2T3) was used. As a control, P. aeruginosa PA14 carrying RK2 was used. The experimental methods described in Example 2 were used. For selection of plasmids, the growth medium was supplemented with 50 μg/ml tetracycline.

The following doses and conditions were used:

| Number of mice | breed | pathogen | dose/cfu |
|---|---|---|---|
| 8 | Swiss Webster | PA14/RK2 | 10⁷ |
| 7 | Swiss Webster | PA14/pCON22-62 | 10⁷ |
| 7 | Swiss Webster | PA14/pCON22-62 | 10⁸ |

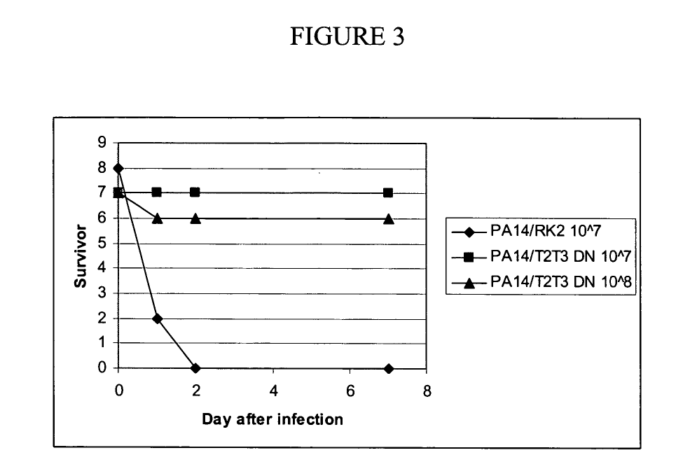

The results are shown in FIG. 3. The animals harboring only the RK2 plasmid were killed very quickly after infection at the dose of 1×10⁷ cfu. This is consistent with the results shown in Example 3, in which the RK2 plasmid was shown not to alter the virulence or PA14. In contrast, the animals infected with PA14 containing the mutant genes (T2-T3 mutations) survived for 7 days, with the exception of one animal, demonstrating that the presence of a plasmid comprising the T2 and T3 mutations attenuates the virulence of PA14 at least 10 fold.

### EXAMPLE 5

### Virulence of Strains Containing Single Mutant Attenuator Plasmids

In this example, the effect of dominant negative mutants of pscN (PA14/pCON22-98, T2 mutant) and xcpR (PA14/pCON22-35, T3 mutant) were tested independently for the ability to attenuate virulence of P. aeruginosa PA14. The double mutant attenuator plasmid PA14/pCON22-62 (T2T3 mutant) and RK2 plasmid were used as controls. The experimental methods described in Example 2 were used. For selection of plasmids, the growth medium was supplemented with 50 μg/ml tetracycline.

The following doses and conditions were used:

| Number of mice | breed | pathogen | dose/cfu |
|---|---|---|---|
| 10 | Swiss Webster | PA14/RK2 | 10⁷ |
| 10 | Swiss Webster | PA14/pCON22-98 | 10⁷ |
| 10 | Swiss Webster | PA14/pCON22-35 | 10⁷ |
| 10 | Swiss Webster | PA14/pCON22-62 | 10⁷ |

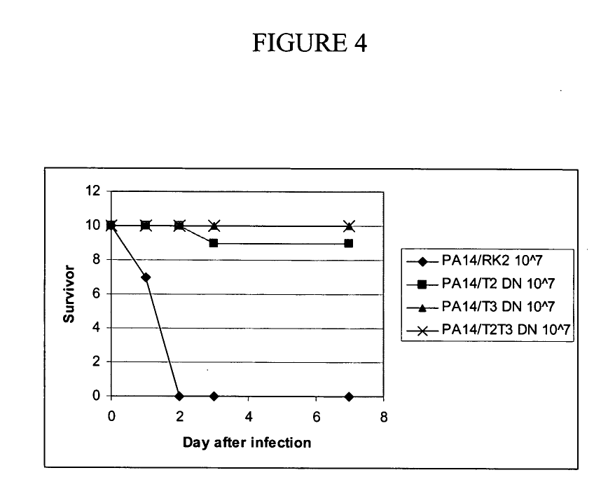

The results are shown in FIG. 4. The animals infected with PA14 carrying only RK2 plasmid were killed quickly (within 2 days). However, all the animals except one survived for 7 days when the pathogens were harboring attenuating plasmids comprising the single dominant negative mutants. With this dose (1×10⁷ cfu), the plasmids carrying either single dominant negative mutant (T2 or T3) attenuate the virulence of PA14 at an equal level to the plasmid carrying the both mutant genes (T2T3).

### EXAMPLE 6

### Virulence of Strains in a Different Breed of Animal

It was next determined whether the pathogen PA14 may show reduced virulence in a different breed of mice, and, similarly, whether the attenuation effect of the dominant negative mutants may be altered. C3H/HeJ is a mouse breed lacking Toll-Like-Receptor 4 (TLR4), resulting in this animal being generally more sensitive to bacterial infection. As a control C3H/HeN was used as a control, as this breed is very similar to C3H/HeJ, but possesses a functional TLR4. The experimental methods described in Example 2 were used. For selection of plasmids, the growth medium was supplemented with 50 μg/ml tetracycline.

The following doses and conditions were used:

| Number of mice | breed | pathogen | dose/cfu |
|---|---|---|---|
| 4 | C3H/HeJ | PA14/RK2 | 10⁷ |
| 4 | C3H/HeJ | PA14/pCON22-62 | 10⁷ |
| 5 | C3H/HeN | PA14/RK2 | 10⁷ |
| 5 | C3H/HeN | PA14/pCON22-62 | 10⁷ |

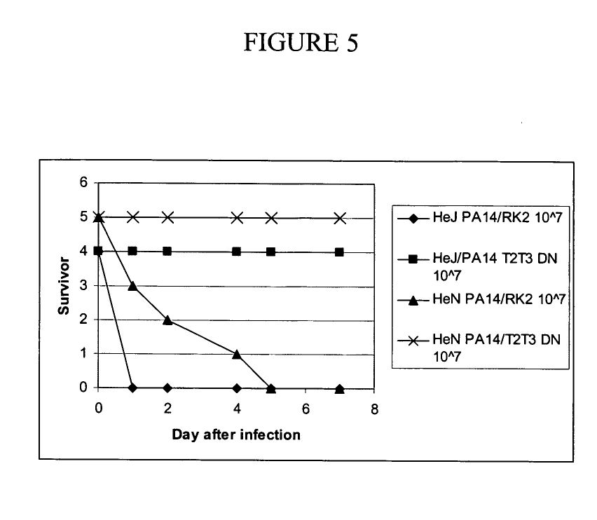

The results are shown in FIG. 5. The survival curve of C3H/HeN suggests that this breed is more resistant to P. aeruginosa PA14, since they were not killed as quickly as other breeds (e.g., Swiss Webster). However, the TLR4-minus breed, C3H/HeJ was at least as sensitive to this P. aeruginosa PA14 as Swiss Webster as they were all killed within 2 days. This provides evidence that the attenuation is not breed specific.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A method for treating a recipient cell, comprising exposing said recipient cell to a donor cell, wherein said donor cell is configured to conjugatively transfer a recombinant transmissible plasmid to said recipient cell, and wherein said recombinant transmissible plasmid is configured to express a gene encoding a protein that alters the function of at least one secretion system in said recipient cell, thereby altering the virulence of said recipient cell.

2. The method of claim 1, wherein said secretion system is selected from a group consisting of type II, type III, and Type IV secretion systems.

3. The method of claim 1, wherein said recipient cell is a pathogen cell, and wherein said altering the virulence of said recipient cell comprises attenuating the virulence of said pathogen cell.

4. The method of claim 1, wherein said donor cell comprises one or more transfer genes.

5. The method of claim 4, wherein said one or more transfer genes are contained on said recombinant transmissible plasmid.

6. The method of claim 1, wherein said recipient cell is a bacterium.

7. The method of claim 6, wherein said bacterium is a Gram-negative bacterium.

8. The method of claim 6, wherein said bacterium is a Gram-positive bacterium.

9. The method of claim 1, wherein said donor cell is a bacterium.

10. The method of claim 1, wherein said donor cell is a non-dividing cell.

11. The method of claim 1, wherein said recombinant transmissible plasmid comprises nucleic acid encoding at least one dominant negative form of a protein involved in said secretion system.

12. The method of claim 11, wherein said nucleic acid encodes a dominant negative pscN protein.

13. The method of claim 11, wherein said nucleic acid encodes a dominant negative xcpR protein.

14. The method of claim 11, wherein said nucleic acid encodes dominant negative pscN and dominant negative xcpR proteins.

15. The method of claim 12, wherein said nucleic acid encodes a K176G dominant negative form of a pscN protein.

16. The method of claim 13, wherein said nucleic acid encodes a G268A dominant negative form of a xcpR protein.

17. The method of claim 1, wherein said nucleic acid encodes at a protein comprising at least one mutation in a Walker box A motif.

18. The method of claim 1, wherein said recombinant transmissible plasmid is selected from the group consisting of pCON22-98, pCON22-35 and pCON22-62.

19. The method of claim 1, wherein said recombinant transmissible plasmid comprises nucleic acid encoding at least one protein that inhibits a regulatory protein involved with said secretion system.

20. The method of claim 3, wherein said pathogen cell is of a genus selected from the group consisting of Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium, Treponema, Borrelia, Leptospira, and Chlamydiae.

21. The method of claim 3, wherein said pathogen cell is selected from the group consisting of Vibrio harveyi, Vibrio cholerae, Vibrio parahaemolyticus, Vibrio alginolyticus, Pseudomonas phosphoreum, Yersinia enterocolitica, Escherichia coli, Salmonella typhimurium, Haemophilus influenzae, Helicobacter pylori, Bacillus subtilis, Borrelia Burgdorferi, Neisseria meningitidis, Neisseria gonorrhoeae, Yersinia pestis, Campylobacter jejuni, Deinococcus radiodurans, Mycobacterium tuberculosis, Enterococcus faecalis, Streptococcus pneumoniae, Streptococcus pyogenes and Staphylococcus aureus.

22. A composition comprising a donor cell comprising a recombinant transmissible plasmid, wherein said recombinant transmissible plasmid comprises nucleic acid encoding a dominant negative form of at least one protein involved a secretion system, and wherein said donor cell is configured to conjugatively transfer said recombinant transmissible plasmid to a recipient cell.

23. The composition of claim 22, wherein said secretion system is selected from a group consisting of type II, type III and Type IV secretion systems.

24. The composition of claim 22, wherein said plasmid is self-transmissible.

25. The composition of claim 23, wherein said nucleic acid encodes a dominant negative pscN protein.

26. The composition of claim 23, wherein said nucleic acid encodes a dominant negative xcpR protein.

27. The composition of claim 23, wherein said nucleic acid encodes dominant negative pscN and dominant negative xcpR proteins.

28. The composition of claim 23, wherein said nucleic acid encodes a protein comprising at least one mutation in a Walker box A motif.

29. The composition of claim 22, wherein said transmissible plasmid is selected from the group consisting of pCON22-98, pCON22-35 or pCON22-62.

30. A system comprising a material in contact with a donor cell, wherein said donor cell is configured to conjugatively transfer a recombinant transmissible plasmid to a recipient cell, and wherein said recombinant transmissible plasmid is configured to express a gene encoding a protein that alters the function of at least one secretion system in said recipient cell, thereby altering the virulence of said recipient cell.

31. The system of claim 30, wherein said secretion system is selected from the group consisting of type II, type III and Type IV secretion systems.

32. The system of claim 30, wherein said material comprises a medical device.

33. The system of claim 30, wherein said material comprises a pharmaceutically acceptable compound.
